(19) United States
(12) Patent Application Publication
(10) Pub. No.: US 2024/0024125 A1
(43) Pub. Date: Jan. 25, 2024

(54) Clamp for Cementing a Prosthesis to a Patella During TKA

(71) Applicant: Brandon Lee Thompson, Pearland, TX (US)

(72) Inventor: Brandon Lee Thompson, Pearland, TX (US)

(21) Appl. No.: 18/356,720

(22) Filed: Jul. 21, 2023

Related U.S. Application Data
(60) Provisional application No. 63/369,140, filed on Jul. 22, 2022.

(51) Int. Cl. A61F 2/46; A61F 2/38

(52) U.S. Cl. CPC A61F 2/46; A61F 2/3877; A61F 2002/4631

(57) ABSTRACT

A lightweight, low profile, disposable clamp for maintaining a compressive force on a cemented patella/patellar prosthesis combination so that the cement interface is not compromised while the cement cures during a total knee replacement.

### CROSS REFERENCE TO RELATED APPLICATIONS

This application for patent claims benefit of and priority to United States Non-Provisional application filed on Jul. 22, 2022, and having Ser. No. 63/369,140, the contents which are incorporated herein by reference for all purposes.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

Field of the Invention. The inventions disclosed herein relate to clamps and similar devices used during a total knee arthroplasty to hold or compress a patellar prosthesis and resurfaced patella in position while bone cement cures there between.

Description of the Related Art Total knee arthroplasty (i.e., TKA) involves three components: a femoral component, a tibial component, and a patellar component and numerous surgical tools. For the patellar component, the posterior surface of the existing patella (i.e., the articular side; the side that previously engaged the natural femoral condyles) is resurfaced, such as by sawing, to reduce the thickness of the existing patella and to form a typically planar surface. A patellar prothesis is provided for the total knee replacement and has a posterior or articular surface designed to operatively engage with the prosthetic “condyles” and an anterior surface designed to mate with, couple to, or engage the resurfaced portion of the existing patella.

Many patellar prostheses are secured to the resurfaced patella with adhesive, such as bone cement. Because bone cement requires time to cure or set (for example, 10-20 minutes), a patellar clamping tool is used to apply a compressive force to the patellar prothesis/patella combination to prevent the interface from moving or loosening while the bone cement sets or cures. While the bone cement is setting (and the patellar clamping tool is applied), the knee replacement surgery may be interrupted or at least delayed because of the existence, size, weight, and location of the clamping tool. Further, because clamping tools are typically made of metal, there is a possibility that the tool could scratch, nick, or otherwise damage the femoral or tibial protheses.

U.S. Pat. No. 10,034,774 is entitled Disposable Modular Patella Protector/Clamp and discloses “[a] disposable modular patella protector and clamp [ ], providing the ability for a surgeon to shift the procedure sequence of the implantation of the patella from the end of a total knee replacement procedure to anytime during the procedure by providing a modular clamp that holds and protects the patella prosthesis and prepared patella. The clamp may be positioned in a manner in which it does not obstruct the patella or the prosthesis, allowing the surgeon to attend to other elements of the procedure while the bone cement is allowed to cure.”

U.S. Pat. No. 10,034,679 is entitled Artificial Prosthesis Installation Clamp and Method and discloses “[i]n the use of devices and methods for measuring applied pressure on an article, such as a patella construct during a surgical procedure, a clamp is provided with arms, an optional external spring, an optional internal spring and a torque knob for applying constant and measurable pressure to the article. Embodiments of the clamp include a spring rod engaged with the torque knob for assisting in applying and retaining desired pressure. Spike elements and a bushing insert are secured to the ends of the arms for retaining the article in place during use. The elements are advantageously assembled and disassembled to facilitate thorough and quick cleaning.”

The present inventions are directed to improved patellar clamps that are small, light weight, and do not interfere with the knee replacement surgery while the clamp is in use.

### BRIEF SUMMARY OF THE INVENTION

To the extent that the inventions disclosed and enabled herein can be briefly summarized, one of many possible brief summaries may be: A clamp for compressing a cemented patella/prosthesis combination may comprise a first member having a surface adapted to contact an anterior surface of a resurfaced patella; a second member that is separable from the first elongated component and that has a surface with an artifact shaped to engage at least a portion of an articular surface of a patellar prothesis, when the prosthesis is cemented to the resurfaced patella; a tension component coupled to one end or edge of the first or the second member, such that the tension component extends away from the member substantially perpendicularly to the surface of the member to which the tension component is coupled; a retention mechanism associated with the member that does not have the tension component coupled thereto, wherein the retention mechanism comprises an interior surface that engages an exterior surface on the tension component; and wherein when the first member contacts the anterior surface of the resurfaced patella and when the second member engages the articular surface of the patellar prothesis, and when a compressive force is applied between the first and second members, at least a portion of the applied compressive force is retained in the clamp by the retention mechanism reacting against the tension member to lock the retained compressive force across the cemented patella/prothesis combination. Other brief summaries are likely possible.

This proposed brief summary of the inventions is not intended to limit or otherwise affect the scope of what has been disclosed and enabled or limit or otherwise affect the scope of the appended claims. Nothing stated in this Brief Summary of the Invention is intended as a definition of a claim term or phrase or as a disavowal or disclaimer of claim scope.

### BRIEF DESCRIPTION OF THE SEVERAL VIEWS OF THE DRAWINGS

The following figures form part of the disclosure of inventions and are included to demonstrate further certain aspects of the inventions. The inventions may be better understood by reference to one or more of these figures in combination with the detailed description of certain embodiments presented herein.

FIG. 1 illustrates a side view of a human knee joint.

FIG. 2 illustrates a prior art patellar clamp.

FIGS. 3A and 3B illustrate an embodiment of a patellar compression system utilizing my inventions.

FIG. 4 illustrates another embodiment of a patellar compression system utilizing my inventions.

FIG. 5 illustrates compression retention mechanisms useful with embodiments of my inventions.

FIG. 6 illustrates another embodiment of a patellar compression system utilizing my inventions.

FIG. 7 illustrates another compression retention mechanism useful with embodiments of my inventions.

FIG. 8 illustrates another compression retention mechanism useful with embodiments of my inventions.

FIGS. 9A and 9B illustrate a retention mechanism lock useful with the retention member illustrated in FIG. 8.

FIG. 10 illustrates another patellar compression system utilizing my inventions.

While the inventions disclosed herein are susceptible to various modifications and alternative forms, only a few specific embodiments are shown by way of example in the drawings and are described in more detail below. The figures and detailed descriptions of these embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts illustrated and taught by the specific embodiments.

### DETAILED DESCRIPTION

The Figures described above, and the written description of specific structures and functions below, are not presented to limit the scope of the inventions disclosed or the scope of the appended claims. Rather, the Figures and written description are provided to teach a person skilled in this art how to make and use the inventions for which patent protection is sought.

A person of skill in this art that has benefit of this disclosure will understand that the inventions are disclosed and taught herein by reference to specific embodiments, and that these specific embodiments are susceptible to numerous and various modifications and alternative forms without departing from the inventions we possess. For example, and not limitation, a person of skill in this art that has benefit of this disclosure will understand that Figures and/or embodiments that use one or more common structures or elements, such as a structure or an element identified by a common reference number, are linked together for all purposes of supporting and enabling our inventions, and that such individual Figures or embodiments are not disparate disclosures. A person of skill in this art that has benefit of this disclosure will recognize and understand the various other embodiments of our inventions having one or more of the structures or elements illustrated and/or described in the various linked embodiments. In other words, not all possible embodiments of our inventions are described or illustrated in this application, and one or more of the claims to our inventions may not be directed to a specific, disclosed example. Nonetheless, a person of skill in this art having benefited from this disclosure will understand that the claims are fully supported by the entirety of this disclosure.

People skilled in this art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation, location and from time to time. While a developer's efforts might be complex and time-consuming in an absolute sense, such efforts would be, nevertheless, a routine undertaking for those of skill in this art having benefit of this disclosure.

Further, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the scope of what is claimed.

Reference throughout this disclosure to “one embodiment,” “an embodiment,” or similar language means that a particular feature, structure, or characteristic described in connection with the embodiment is included in at least one of the many possible embodiments of the present inventions. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

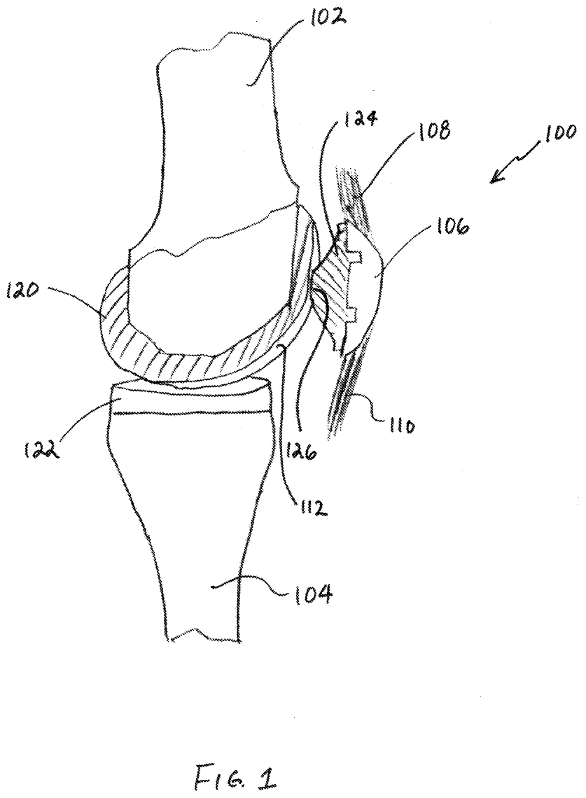

FIG. 1 illustrates the anatomy of a human knee 100 with a total knee replacement showing the femur 102 and femoral component 120, the tibia 104 and tibial component 122, the patella 106 and patellar component 124, and the patellar tendons 108 and 110. Trochlear groove 112, in which the posterior or articular surface 126 of the patellar prosthesis 124 slides, is also shown.

During a total knee replacement, the patella 106 is everted from the body such that the posterior surface is effectively facing up toward the surgeon rather than facing down toward the condyles. The patella 106 may be resurfaced by removing, such as by sawing, the posterior surface 126 to create a more or less planar surface. Holes, for example three, are reamed or drilled into the planar surface to orient the patellar prosthesis 126. For patellar prostheses, e.g., prosthesis 126, that require bone cement to secure the prosthesis to the patella 106, a clamp or other compression device is employed to press the prothesis into the planar surface while the bone cement therebetween cures or hardens.

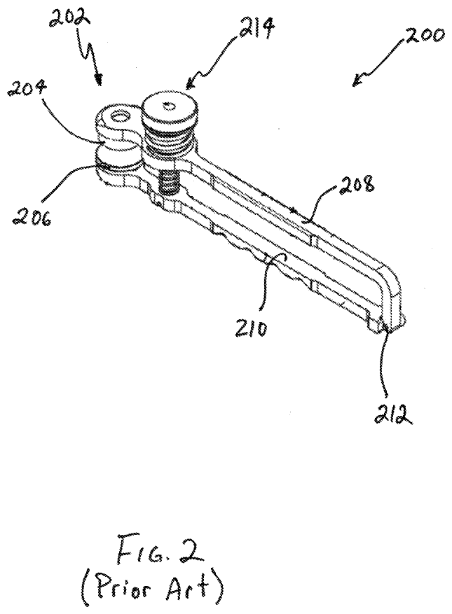

FIG. 2 illustrates a conventional patellar clamp 200, as disclosed in U.S. Pat. No. 10,034,679 having a clamping assembly 202 with a bushing 204 for contacting the patellar prosthesis, and a cap 206 for contacting the anterior surface of the patella. The bushing 204 is coupled to a top arm 208, and the cap 206 is coupled to a lower arm 210. The top arm 208 and the lower arm 210 are joined by a hinge 212, which allows the bushing 204 and the cap 206 to move toward and away from each other. Disposed between the clamping assembly 202 and the hinge is a threaded knob assembly 214 that allows the user to compress the patella/prosthesis combination while the bone cement sets.

With the foregoing as context, I have invented compression systems for use during knee surgery, such as a total knee arthroplasty, when the patella is resurfaced to accept a cemented patellar prosthesis. The compression systems may comprise a patella member optimized in size, shape, and/or material to contact and apply a compressive force to the anterior surface of the patella; a prothesis member optimized in size, shape and/or material to contact and apply a compressive force to the articular surface of the patellar prothesis; a tension component spanning at least between the patella member and the prosthesis member; and a compression retention mechanism operable with the tension component to hold or lock a compression force applied across the members. The tension component may be located adjacent one end of the members such the compression system resembles the letter “C” or a sideways “U.” It is preferred, but not required that the tension component be substantially perpendicular to either or both of the patella member and the prosthesis member. By “substantially perpendicular,” I mean 90°±15°.

The tension component may be implemented as a unitary structure or as a plurality of structures. The tension component may be formed or made integral with either the patella member or the prosthesis member, or the tension component may be a separate structure or structures from the members. The compression retention mechanism(s) may be formed or made integral with the members, or the compression retention mechanism(s) may be separate structures from the member and tension component(s). The compression retention mechanism may include but does not have to include a release or unlock mechanism.

The compression systems may be fabricated from any medical grade material, including metals, polymers (plastics), ceramics, composites, and combinations thereof. It is preferred that the compression system be fabricated from a lightweight material that will not harm or scratch the other knee replacement components, such as the femoral and tibial components. It is also preferred that the compression system be a one-time use, disposable component, which purpose may inform the material selection. It is also preferred that the compression system have a low profile.

The compression system may be removed from the patella/prosthesis combination, such as when the bone cement has cured, set, or hardened, by releasing or unlocking the retention mechanism to remove the applied compression, or by physically spreading the members (and potentially elastically deforming the tension component) to dislodge the compression system from the patella/prosthesis combination.

In use, the compression system may be slid or installed over a patella/prosthesis combination such that the prosthesis member contacts the articular surface 126 of the prosthesis 124, and the patella member contacts the anterior surface of the patella 106. The user may then squeeze or compress the members, manually or with a tool, to apply a compressive force to the patella/prosthesis combination. The compression retention mechanism is structured to resist releasing the compressive force to maintain the integrity of the cemented interface while the bone cement cures. When the bone cement has cured, the release mechanism, if utilized, may be activated, such as by depressing or pulling with a finger or an instrument, to release the retention mechanism and thereby release the compressive force. Alternately, the members may be spread apart (such as by elastically or plastically deforming the tension component) and removed from the patella/prothesis combination. The compression system may then be removed from the patient and discarded or collected for sterilization and reuse.

It is preferred, but not required, that any exposed portion of the tension component extends outwardly from the everted patella toward the surgeon rather than inwardly toward the knee. Additionally, in some embodiments, it may be preferred to fabricate the tension components from a material that can be cut so that the extending portions may be removed after the compression force has been applied.

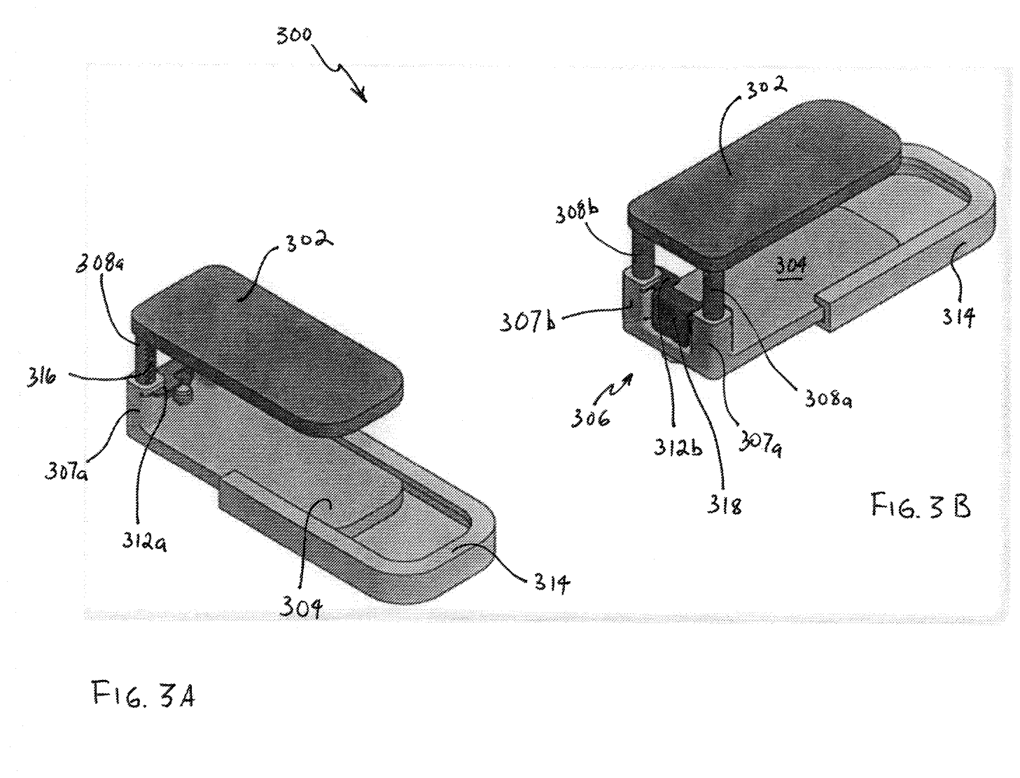

Turning now to a description of some, but not all, possible embodiments of my inventions, FIGS. 3A and 3B disclose a compression system 300 comprising a patella compression member 302 and a separate prosthesis compression member 304. In this embodiment, the prosthesis member 304 further comprises a retention mechanism 306 having receptacles 307a and 308b formed integrally with the member 304, a pair of biased dogs 312a, 312b and a release mechanism 318. It is preferred that the patella and the prosthesis members have a width of between about 0.75 inches and about 1.5 inches. In some embodiments, the patella member may be wider than the prosthesis member.

The patella member 302 comprises a pair of tension components 308a and 308b, which are sized and shaped to operatively engage the respective receptacles 307a and 307b. Each tension component 308 comprises a plurality of slots 316 formed on the outer surface of the tension component 308. It is preferred that the plurality of slots 316 be spaced to accommodate the thickness of most patella/prothesis combinations, which range from about 0.6 inches (15 mm) to about 1.2 inches (30 mm).

As illustrated, the dogs 312 are biased into slots 316 in the tension components 308 to lock the members, 302, 304 in relative position. The release 318 can be pressed to move the dogs 312 out of locking engagement with the slots 316.

In use, the compression system 300 may be placed or slid over a patella/prosthesis combination such that the prosthesis member 304 contacts the articular surface 126 of the prosthesis 124, and the patella member 302 contacts the patella 106. The user may then depress the release 318 to move the dogs out of the way and manually squeeze or otherwise compress the members 302, 304 to apply a compressive force to the patella/prosthesis combination having bone cement at the interface. Alternately, the slots 312 may be structured so that they only lockingly engage the dogs when the members 302, 304 are attempting to move away from each other. For tension components having unidirectional locking slots 316, the use need not depress the release 318 while squeezing the members. 302, 304. When the bone cement has cured or when the compression system 300 is desired to be removed, the release 310 may be activated, such as by depressing with a finger or an instrument, to release the dogs 312a and 312b and thereby release the compressive force. The compression system 300 may then be removed from the patient and discarded or collected for sterilization and reuse.

Depending upon the material from which the members 302, 304 are fabricated, the members, such as the prosthesis member 304, may slide or move with respect to the articular surface 126 of the prosthesis 124. To prevent or retard such movement, the compression system 300 may comprise a sleeve 314 sized and shaped to slide over the prosthesis member 304 to create a groove or channel to restrain the prothesis against movement. Alternately, the prosthesis member 304 may comprise a depression, groove, channel or other surface feature formed into or on the member 304 to prevent or reduce movement of the patella/prosthesis combination with the compression system 300.

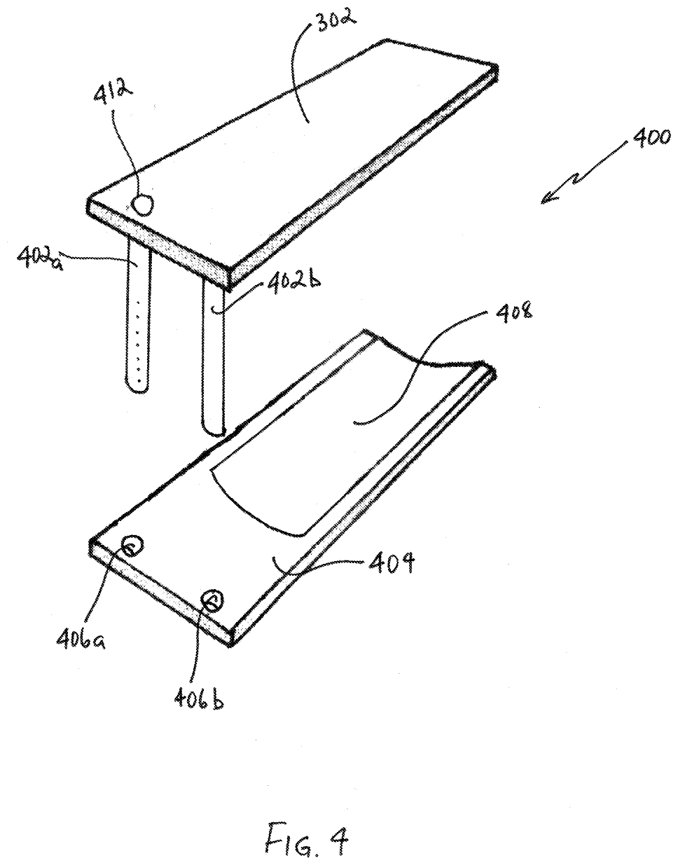

FIG. 4 illustrates another embodiment of a compression system 400 comprising a patella member 302 having a pair tension components 402a and 402b. For illustration purposes, tension component 402a is shown as a separate structure that has been secured 412, such as by gluing or welding, to member 302. Tension component 402b is illustrated to be formed integral with member 402. Although not shown, the surface of member 402 that contacts the patella 106 may have a surface treatment to reduce movement or increase gripping of the patella during use.

System 400 also comprises a prosthesis member 404 having a surface feature for engaging the articular surface 126 of the prosthesis 124. FIG. 4 illustrates a groove or guide 408 for the articular surface 126 of the prosthesis 124. The member 404 also comprises receptacles 406a and 406 into which the distal portions of the tension components 402a and 402b may be received.

Similar to the system 300, the system 400 may comprise a retention mechanism to lock in the compressive force. One type of retention system that may be used with this and other embodiments comprises spaced holes 410 in the tension components 402. It will be appreciated that the holes 410 are preferably spaced at discrete intervals along the tension components to accommodate patella/prosthesis combination of varying thicknesses.

In use, once the user has placed the system 400 on the patella/prosthesis combination, the user may manually squeeze or use a tool to squeeze the system 400 to apply compression to the combination. To retain the compression, the user may simply insert a suture needle into the appropriate hole in each tension component so that the body of the needles react against a surface of the member 404 to retain the compressive force To release the compression and remove the system 400, the user simply has to remove the suture needles such as with a forceps or hemostat.

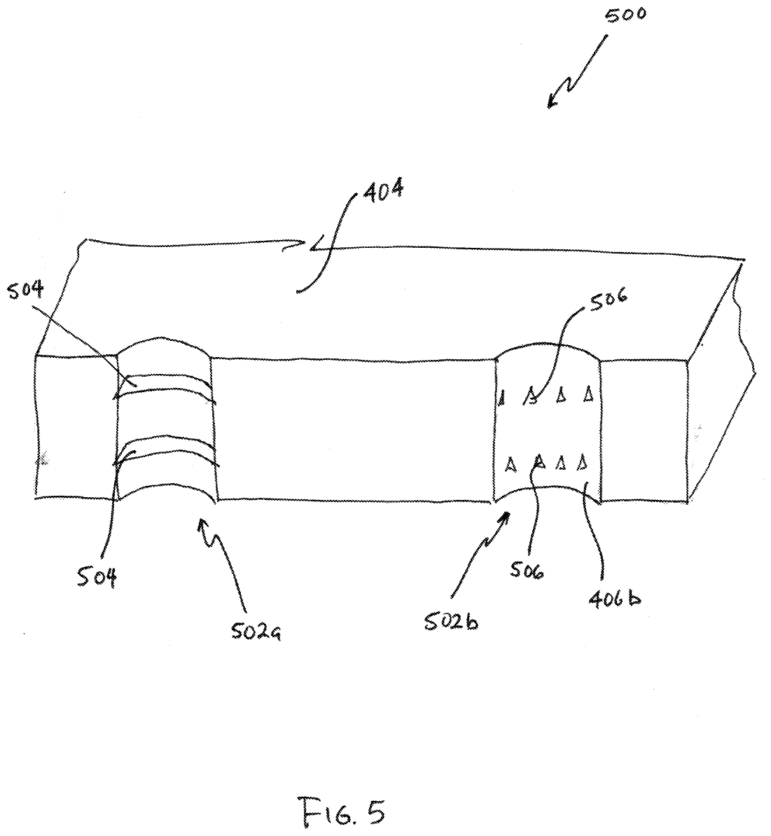

FIG. 5 illustrates an alternate retention mechanism 500 useful with the system 400 and other embodiments. In FIG. 5, retention mechanism 500 is made a part of the prosthesis member 404, and specifically, is integrated into the receptacles 406a and 406b. In this embodiment, receptacle 406a is illustrated to comprise one or more fully or partially toroidal rings 504 embedded in the receptacle 406a such that the exposed ring edges 504 engage the tension component 402a and resists, prevents or retards movement of the tension component that would release the applied compressive force.

Receptacle 406b illustrates an alternate embodiment in which a plurality of individual, embedded barbs 506 are disposed on the inner surface of the receptacle 406b such that the barbs allow the tension component 402b to pass through in one direction but not pass in the opposite direction, which opposite direction would release the applied compressive force.

In use, a compression system utilizing a retention mechanism 500, such as compression system 400, may be removed from the patient by simply spreading the members away from each other thereby elastically and/or plastically deforming (e.g., bending) the tension components 402a and 402b to allow the system to be removed. It is contemplated that for embodiments without a release mechanism, a tab, ring, or other structure may be provided at the end of member 404 opposite the tension components 402 to aid grasping and spreading the members 402, 404.

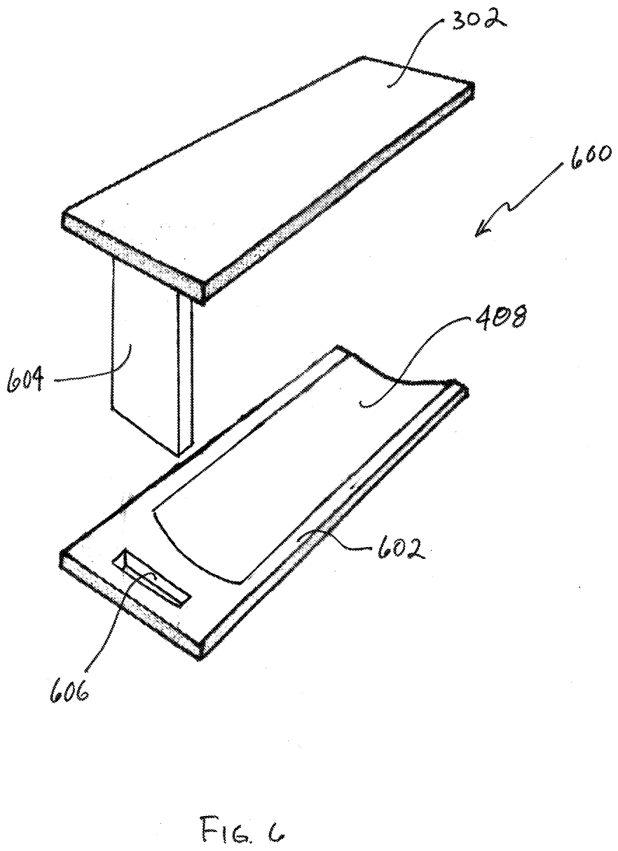

FIG. 6 illustrates another possible embodiment of a compression system 600 comprising a patella member 302 and a prosthesis member 602, which member 602 shows an optional prosthesis groove 408. Member 302 may comprise a unitary tension member 604 disposed adjacent one end of the member 302. Member 602 comprises a corresponding receptacle 606 structured to receive the tension member 604. In this embodiment, the system 600 also may comprise a retention mechanism, such as one or more of the retention mechanisms disclosed in FIGS., 3, 4 and/or 5.

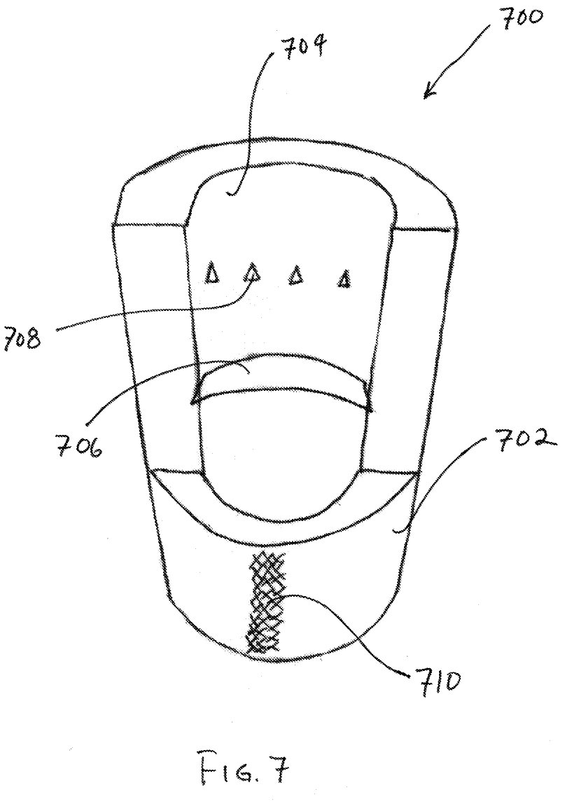

FIG. 7 illustrates an alternate retention member 700 that may be used with any of the embodiments disclosed or other embodiments. Retention member 700 comprises a body 702 comprising an inner surface 704 and an outer surface. The inner surface 704 may comprise one or more or a combination of movement restriction devices, such as rings 706 or barbs 706.

It will be appreciated that the retention member 700 may be formed integrally with or secured to a member, preferably the prosthesis member, such as illustrated in FIGS. 3A and 3B. In use, as the user squeezes or clamps the compression system, the retention member 700 grips the tension component and restricts movement to the patella or prosthesis members that would reduce or eliminate the applied compressive force.

Alternately, retention mechanism 700 may be a structure independent of the member, and in that iteration may take the form of a push nut. In use, after the user has squeezed or clamped the compression system, the retention member 700 may be pushed down the exposed tension component until the retention member contacts the patella or prosthesis member to retain the compressive force.

Furthermore, the member 700 may comprise internal threads that cooperate with corresponding threads on the tension members. In use, after the user has squeezed or clamped the compression system, the threaded retention member 700 may be screwed on to the exposed tension component until the threaded retention member 700 contacts the patella or prosthesis member to retain the compressive force. Exterior surface treatment, such as knurling, may benefit the retention members 700.

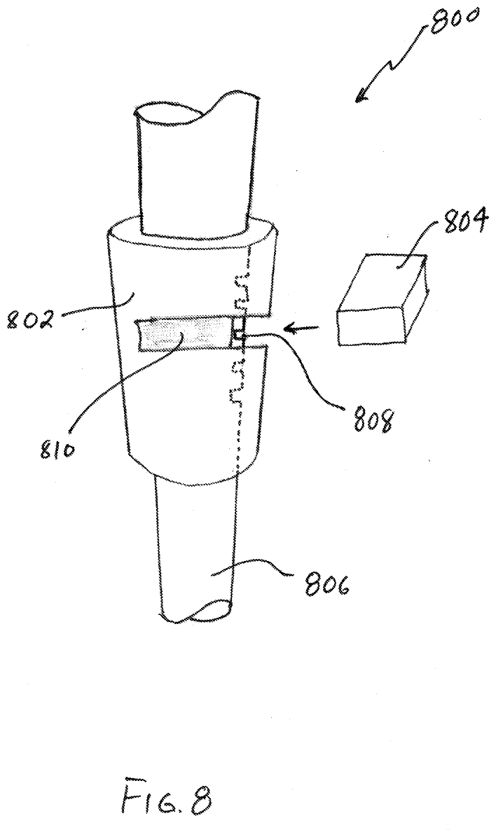

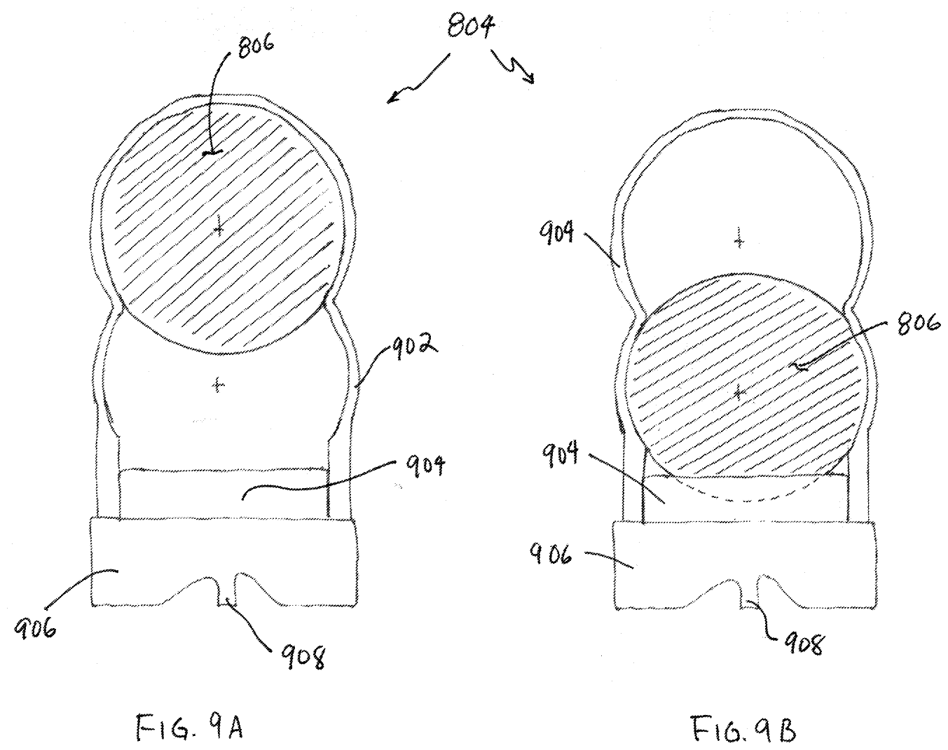

FIGS. 8, 9A and 9B illustrate another embodiment of a retention mechanism 800. Retention mechanism 800 comprises a retention member 801 and a compression lock 804. Retention member 801 comprises a body 802 through which a tension component 806 may pass. The tension component 806 may comprise a plurality of slots 808 into which the compression lock 804 may engage. FIGS. 9A and 9B illustrate a preferred compression lock 804 comprising a body 902 defining first and second tension member 806 positions. The lock 804 comprises a dog 904, such as an elongated tang sized and shaped to operatively engage with the slots 808 (one at a time) to lock the members in relative position. The lock 804 also comprises a handle 906 and, optionally, a removal tab 910. In this embodiment, the retention member 802 and compression lock 804 are a combined unit to form the retention mechanism 800 such that the compression lock 804 is not separable from the retention member 802. Optionally, retention member 801 may comprise an outer surface guide or groove in the compression lock 804 can transit from the unlocked to the locked condition.

FIG. 9A shows the lock 804 in the unlocked condition in which the tension member 806 is free to slide within the retention member 801 without restriction. FIG. 9b shows the lock 804 in the locked condition in which the tension member 806 is restrained within the retention member 801 by the dog 904 engaging a slot 808 to retain the applied compressive force.

The retention mechanism 800 may be formed integrally with the patella/prosthesis member, in which case after the user has operably installed the system on the patella/prosthesis combination and compressed the same, the user may slide the compression lock 804 from the unlocked to the locked condition. When desired, the user may unlock the compression lock 804 by sliding it to the unlocked condition.

Alternately, the retention mechanism(s) 800 may a separate structure from the patella and prosthesis member, in which case in which case after the user has operably installed the system on the patella/prosthesis combination and compressed the same, the user may slide the compression lock 804 to the appropriate position on the exposed portion of the tension component and active the compression lock so that the dog 904 engages a slot 808 in the one or more tension components, thereby retaining the applied compressive force. The compressive force may be released by moving the lock 804 in the opposite direction.

While the foregoing embodiments are illustrated for simplicity with rectilinearly shape components, it is contemplated that either or both of the patella and prothesis members may be rounded, curved or otherwise shaped to better engage and evenly compress the cemented patella/prosthesis combination.

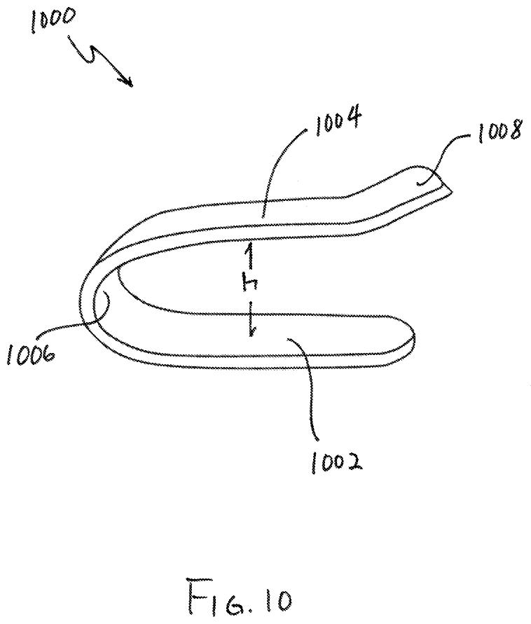

FIG. 10 illustrates a further embodiment of my inventions comprising a unitary compression system 1000 comprising a first portion 1002 sized and shaped to engage a patella 106, a second portion 1004 sized and shaped to engage an articular surface 126 of a prothesis 124, and a portion 1006 disposed between portions 1002 and 1004. The second portion 1004 may comprise an articular surface 126 artifact (not shown) as previously disclosed. The system 1000 may constructed to have a distance “h” between the patella prothesis portion 1004 and the patella portion 1002 sized for the specific patella/prothesis combination employed in the TKA. For example, the distance “h” of the system 1000 should be less than the distance between the outer most surface of the patella/prosthesis combination. The joining portion 1006 preferably is resilient or elastic such that when the system 1000 is engaged on the patella/prosthesis combination having a height to thickness greater than “h,” the combination is compressed while the bone cement cures. The system 1000 may comprise a portion 1008 that enables removal of the system 1000 by a user's finger or tool. While it is preferred that the system 1000 be fabricated from a medical grade polymer and be single use, it is contemplated the patella prothesis portions may be metal and the joining portion 1006 may be a polymer, or vice versa.

Those persons of skill having the benefit of this disclosure will now understand and be enabled to produce embodiments of my inventions using one or more of or combining aspects of one or more of the features in the previously disclosed embodiments and those features and aspects taught by these embodiments.

Other and further embodiments utilizing one or more aspects of the inventions described above can be devised without departing from the spirit of my invention. Further, the various methods and embodiments of the methods of manufacture and assembly of the system, as well as location specifications, can be included in combination with each other to produce variations of the disclosed methods and embodiments. Discussion of singular elements can include plural elements and vice versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by me, but rather, in conformity with the patent laws, I intend to protect fully all such modifications and improvements that come within the scope or range of equivalent of the following claims.

What is claimed:

1. A clamp for maintaining pressure across a prosthesis cemented to a resurfaced patella, comprising:
a first elongated member having a surface adapted to contact an anterior surface of the resurfaced patella;
a second elongated member independent of the first elongated member and having a surface with a groove therein to engage at least a portion of an articular surface of the prothesis;
a pair of tension components secured to one end of the second elongated member and spaced apart from one another, each tension component extending substantially perpendicularly away from the second elongated member, each tension component having a plurality of slots formed along an outer surface;
a pair of retention members coupled to the first elongated member at one end of the first elongated member and spaced apart from one another such that the each of the tension components can be received in a corresponding retention member at the same time;
a lock operatively coupled with the pair of retention members and comprising a pair of dogs sized and shaped to engage the slots in the pair of tension components to thereby hold the first and second members in relative position to another;
a release mechanism operatively coupled to the lock and comprising a spring that biases the pair of dogs into corresponding slots on the pair of tension components and which spring may be overcome to release the pair of dogs from the engaged slots; and
wherein when the clamp is placed about the cemented prosthesis/patella combination, and when a compressive force is applied to the first and second members, at least a portion of the applied compressive force is retained in the clamp by the pair of dogs engaging a pair of slots.

2. The clamp of claim 1, wherein the pair of tension components have slots placed along the tension components sufficient to clamp cemented prosthesis/patella combinations having thicknesses between 0.6 inches and 1.2 inches, inclusive.

3. The clamp of claim 2, wherein the elongated members, tension components and retention members are fabricated from medical grade polymer.

4. A clamp for compressing a cemented patella/prosthesis combination, comprising:
a first member having a surface adapted to contact an anterior surface of a resurfaced patella;
a second member separable from the first elongated component and having a surface with an artifact shaped to engage at least a portion of an articular surface of a patellar prothesis, which prosthesis is cemented to the resurfaced patella;
a tension component coupled to one end or edge of the first or the second member, the tension component extending away from the member substantially perpendicularly to the surface of the member to which the tension component is coupled;
a retention mechanism associated with the member that does not have the tension component coupled thereto, the retention mechanism comprising an interior surface that engages an exterior surface on the tension component; and
wherein when the first member contacts the anterior surface of the resurfaced patella and when the second member engages the articular surface of the patellar prothesis, and when a compressive force is applied between the first and second members, at least a portion of the applied compressive force is retained in the clamp by the retention mechanism reacting against the tension member to lock the retained compressive force across the cemented patella/prothesis combination.

5. The clamp of claim 4, further comprising a release mechanism operatively coupled to the retention mechanism to selectively release the retained compressive force.

6. The clamp of claim 5, wherein the tension component comprises a plurality of spaced apart slots along a length of the tension component, wherein the retention mechanism comprises a dog sized and shaped to engage the plurality of slots, and wherein the release mechanism comprises a spring that biases the dog into one of the plurality of slots, and wherein the spring can be overcome to release the dog from the one slot.

7. The clamp of claim 6, wherein the plurality of slots are shaped such that the dog engages a slot and restrains movement of the tension component in only one direction.

8. The clamp of claim 7, wherein each member is elongated, and wherein the tension component is coupled to the member adjacent to an end of the elongated member.

9. The clamp of claim 8, wherein the tension component has slots placed along the tension component sufficient to clamp cemented patella/prosthesis combinations having thicknesses between 0.6 inches and 1.2 inches, inclusive.

10. The clamp of claim 9, wherein the tension component is coupled to and adjacent an end of the second elongated member, and wherein the retention mechanism is coupled to and adjacent an end of the first elongated member.

11. The clamp of claim 10, wherein the surface artifact is a depression formed in the second elongated member in which at least a portion of the articular surface of the prosthesis may reside.

12. The clamp of claim 11, wherein the surface artifact is a groove formed in the second elongated member in which the articular surface of the prosthesis may reside.

13. The clamp of claim 11, wherein the tension component comprises first and second tension components, wherein the retention mechanism comprises first and second retention mechanisms are shaped to receive, respectively, first and second tension components, and wherein the release mechanism is disposed between the first and second retention mechanisms.

14. The clamp of claim 4, wherein the retention mechanism comprises a receptacle sized and shaped to receive the tension component and one or more unidirectional restraint devices in the receptacle that engage the tension component to retard or prevent relative movement of the tension component in one direction.

15. The clamp of claim 14, wherein the retention mechanism is a structure independent of the first or second members.

16. The clamp of claim 15, wherein the retention mechanism comprises a push nut.

17. The clamp of claim 16 wherein the one or more restraint devices is a barb or an edge that deforms an outer surface of the tension component.

18. The clamp of claim 14, wherein the retention mechanism is integrally coupled to the one of the first or second members.

19. The clamp of claim 18 wherein the one or more restraint devices is a barb or an edge that deforms an outer surface of the tension component.

20. The clamp of claim 4, wherein the first and second members can be separated by bending the tension components to remove the clamp from the cemented patella/prosthesis combination.
